(19) United States
(12) Patent Application Publication
(10) Pub. No.: US 2011/0190655 A1
(43) Pub. Date: Aug. 4, 2011

(54) Scanning head including at least two electrodes for impedance measurement, arrangement, and related method

(76) Inventors: Ulrich Moissl, Bad Vilbel (DE); Paul Chamney, Herts (GB); Peter Wabel, Darmstadt (DE)

(21) Appl. No.: 13/058,590

(22) PCT Filed: Aug. 26, 2009
(86) PCT No.: PCT/EP2009/006189

§ 371 Date: Mar. 24, 2011

(30) Foreign Application Priority Data
Aug. 27, 2008 (DE) 10 2008 039 844.6

(51) Int. Cl. A61B 5/053

(52) U.S. Cl. 600/547

(57) ABSTRACT

The present invention proposes a scanning head including at least two electrodes, in particular voltage measurement electrodes, for the impedance measurement on a patient's body. It moreover specifies a corresponding arrangement including a scanning head and suitable methods.

### CROSS REFERENCE TO RELATED APPLICATIONS

This is a 371 national phase application of PCT/EP2009/006189 filed Aug. 26, 2009, claiming priority to German Patent Application No. 10 2008 039 844.6 filed Aug. 27, 2008.

### FIELD OF INVENTION

The present invention concerns a scanning head including at least two measurement electrodes for the impedance measurement on a patient's body. It furthermore concerns an arrangement including a scanning head, a method for measuring an impedance of a body, and a method for deriving voltage differences on the surface of a patient's body.

### BACKGROUND OF THE INVENTION

Bioimpedance measurement or impedance measurement on a patient is known in practice as a method, e.g., for determining the composition of a patient's body, in particular a patient's water content. By means of this method it is possible, e.g., to recognize and/or differentiate changes of a patient's fluid metabolism or nutritional condition and tissue compositions, as is known, e.g., from WO 2006/002685 A1 and U.S. Pat. No. 6,678,552 B2.

Up to now, impedance measurements or bioimpedance measurements have been performed as the measurement of sum impedances or summed impedance changes of individual extremities of the body or portions thereof (arm, lower leg), of the torso, or as a whole-body measurement. In each one of the above-mentioned methods, current is introduced into the body via electrodes, and a resulting voltage is measured on the surface. The measurement results allow a determination of the tissue impedance. The electrodes provided for the introduction of current on the one hand and the electrodes for the voltage measurement on the other hand are affixed on the body by means of corresponding fastening bands, e.g., in the area of the wrist and ankle joint. In the prior art, the electrodes are always arranged in a fixed manner.

Locally increased or reduced impedances as occurring, e.g., in the case of an edema of the lower leg or of a locally limited accumulation of fluid, as in ascites, enter into the result, e.g., of a whole-body impedance measurement. They remain undiscovered owing to their frequently small relative contribution to the summation, may falsify measurement results, or cannot be assigned to single portions of the body with sufficient accuracy. The same is true for local impedance measurements which are also always carried out with fixedly arranged electrodes.

### SUMMARY OF THE INVENTION

It is an object of the present invention to specify further equipment for the performance of an impedance measurement on a patient and corresponding methods.

In accordance with the invention, a scanning head or probe includes at least two electrodes, at least one of which is configured as a measurement electrode, in particular as a voltage measurement electrode, for the impedance measurement on a patient's body. The scanning head may in accordance with the invention include measurement electrodes only or may include current introduction electrodes only. It may moreover include both measurement electrodes and current introduction electrodes. In particular, it may include at least one measurement electrode and at least one current introduction electrode.

In terms of the invention, a scanning head is understood to be a device including the above-mentioned at least two electrodes, which may be moved across a patient's body substantially freely—i.e., independently of location—for measuring or determining an electrical field, a voltage, the impedance and/or respective changes thereof, or for the introduction of current. The kind of free mobility may correspond to that of an ultrasound head utilized by the physician. The scanning head may be coupled to a diagnostic unit by means of wire lines so as to be movable in the degree required for the examination. Instead of the wire lines, however, a non-contact transmission involving a diagnostic unit may equally be carried out. In this case, wire lines between scanning head and diagnostic unit may be unnecessary.

The measurement electrodes or the current introduction electrodes of the scanning head permit a determination or measurement, in particular a voltage determination or measurement, on the surface region of the patient being examined with the aid of the scanning head, the patient being connected to a power source. If the user of the scanning head of the invention moves the scanning head from a first region on the body surface to a second region, the scanning head first measures the impedance of the first region and then the impedance of the second region. Both the first region and the second region may here be situated between two current introduction electrodes. The scanning head is thus configured in such a manner that its electrodes precisely do not remain on the body in a fixedly placed manner, as is the case in the prior art. On the contrary, the scanning head is usable independently of the location on the body of the patient being examined. In accordance with the invention, “independently of location” quite generally is understood to mean that an examination of more than one body region may be carried out without detaching and possibly again fixing electrodes such as current introduction electrodes or measurement electrodes on the body being examined by means of adhesive connection, velcro tape, rubber band, or the like.

The expression “patient” in the framework of the present invention applies to both humans and animals. Moreover, the present invention may be employed for an impedance measurement on living tissue and post-mortem for the examination of dead tissue by the pathologist or forensic examiner.

Thus it is proposed in a preferred embodiment to configure the scanning head in such a manner that at least two of the scanning head's electrodes may be taken into contact with the same portion of the patient's body. The respective electrodes may be applied concurrently or simultaneously, e.g. on the skin of the abdomen, of the chest, or in the area of a joint of the patient as examples for “the same body portion” for an impedance measurement, in particular also between the two electrodes. The same body portion may in accordance with the invention be understood to be a region including skin areas each being, during utilization of the scanning head, in contact with one of the electrodes which are located only a few centimeters apart in the anatomical neutral-zero position. The expression “same body region” is therefore not used in accordance with the invention if, for instance, an electrode is in contact (which contact should presently in general be understood as an electrical contact) with the skin of the patient's epigastrium, whereas another electrode is in contact with the skin in the area of the patient's hand or even of another person. In accordance with the invention, an electrode placed in contact with the skin of the hand during the examination of the patient by means of the scanning head of the invention is referred to as a hand electrode as known from the prior art, however not as a scanning head electrode within the meaning of the invention.

In another preferred embodiment of the invention it is proposed to arrange at least two of the electrodes of the scanning head at a fixed distance from each other. This distance may be five or ten centimeters, for instance, with other values such as, e.g., between one centimeter and fifteen centimeters also being encompassed by the invention. The three-dimensional resolution ultimately increases with a closer arrangement of the electrodes, whereby the signal strength decreases on the other hand. The distance of the electrodes must therefore be chosen in accordance with the application.

In a particularly advantageous embodiment, the two measurement electrodes may be adapted to be movable relative to each other, and optionally to be immobilized. Here the distance of at least two electrodes of the scanning head may be variable or adjustable relative to each other. This may be realized, e.g., through the intermediary of shifting means for at least one electrode.

In a further preferred embodiment of the present invention, in turn, the scanning head includes at least one means for determining a position of the scanning head on or above the surface of the patient's body.

The means for determining a position of the scanning head may be provided on the scanning head itself. In accordance with the invention it may, however, also be provided on an arrangement containing the scanning head that is equally encompassed by the present invention instead of on the scanning head (or in addition thereto).

The determination of what position the scanning head assumes in a specific impedance measurement relative to the body surface or to a point of reference, allows a comparison of the values measured during this specific impedance measurement with values of a reference population on the same location of the body. In addition it allows comparative, reproducible measurements on the same patient at different times. The latter advantageously permits one to determine changes over time of the impedance and thus of the examined tissue with an accuracy that could previously not be achieved, in particular with regard to a three-dimensional resolution or allocation.

The accurate knowledge of the scanning head's position during the impedance measurement advantageously permits a simplified combination of the results obtained by means of impedance measurement and other methods such as ultrasound, which may be combined into meaningful, three-dimensional representations when having knowledge of the respective position. Furthermore the generation of impedance-tomographic representations, especially with utilization of a plurality of scanning heads, is advantageously possible when having accurate knowledge of the respective position(s) of the scanning head(s) during the measurement.

The position of the scanning head relative to, e.g., hand, arm and leg electrodes as points of reference may be detected automatically in a continuous manner via radio sensors. The system is thus capable of automatically recognizing that, for instance, a measurement is being carried out in the area of the liver. Automatic matching with reference values of healthy persons by comparing the measured impedance with values of comparable surface regions of reference persons are suited to detect deviations of the impedance in the respective body region.

In a further preferred embodiment of the present invention, in turn, a means is provided for determining a differential distance covered by the scanning head between the first and the second positions on the body surface or relative to the body surface or relative to an arbitrary point of reference. In addition, the scanning head may be configured to include means for determining a velocity at which the scanning head is moved across the patient's skin surface.

The distance covered by the scanning head during the examination of the patient may be detected, e.g., with the aid of a roller wheel. In the same way, a detection of the position of the scanning head (also independently of a covered distance) by means of an optical correlation system or by means of radio sensors in order to determine the position is encompassed by the present invention.

The present invention is also achieved through an arrangement including at least one scanning head as well as a bioimpedance measurement apparatus and at least one or two further or additional electrodes, for example current introduction electrodes which preferably are not present in, within, at, or on the scanning head. As the advantages described in connection with the scanning head of the invention may also be achieved undiminished by means of the arrangement of the invention, reference is made to the above discussion thereof so as to avoid repetitions.

The additional electrodes having, e.g., the form of current introduction electrodes, can introduce or tap the current—as is also customary in the prior art—for example in the area of wrist or ankle joint and measure it there. A leg-to-leg, head-to-foot, head-to-hand or hand-to-hand arrangement is, of course, also conceivable.

The present invention is furthermore achieved through the method described herein. As the respective advantages that may be achieved hereby undiminishedly correspond to those that may be achieved with the scanning head and/or the arrangement, express reference is herewith made to their above discussion so as to avoid repetitions.

The scanning head may be used with the aid of a mobile power source—e.g., battery-powered—or powered by a fixed energy unit such as, e.g., the one of the diagnostic unit used.

The electrodes of the scanning head—however, not the additional electrodes—may in accordance with the invention further be provided while being distributed to more than only one scanning head. This permits, e.g., a measurement from abdomen to back or vice versa, as well as an examination of the impedance across larger distances which may nevertheless be determined in a variable manner. In accordance with the invention, a scanning head is therefore also understood to be a multi-part scanning head, single parts of which may be used separately from each other with a respective at least one electrode, in particular a measurement electrode. A scanning head may in accordance with the invention also be understood to be a plurality of scanning heads.

By means of the scanning head of the invention, bioimpedance or impedance (the two expressions may here be understood to be interchangeable) can be measured at a single frequency (bioimpedance analysis). It is, however, also possible to perform a spectroscopy for determining a locus curve.

By means of the scanning head of the invention it is advantageously possible to selectively measure or detect, e.g., cardiac pulsation by means of an impedance measurement, for instance on one of the large arterial vessels. In the same way, the inclusion of water in the lungs may advantageously be detected. Both of these are measurements that are not possible in the prior art owing to the invariably fixed placement of the electrodes.

A classification of fat tissue into subcutaneous fat or visceral fat is advantageously possible by means of the scanning head of the invention thanks to its suitability for being placed locally, or the locally performed measurement of the impedance, respectively. A left/right comparison may also be performed in a purposeful manner. The scanning head of the invention is furthermore suited for use in the rehabilitation of a patient in rebuilding muscle, for instance following joint surgery. It is suited for the recognition of lymphatic edemas and may be employed both lying down and standing up. A combination of its utilization with weighing of the patient is also possible in accordance with the invention. Thus, the results with regard to impedance that were obtained by means of the scanning head may be incorporated in bioimpedance model equations wherein the weight is equally of relevance, in order to obtain statements that are relevant in terms of health.

By a brushing or rolling movement of the scanning head on the body, e.g., from the neck down to the foot, it is possible to generate and optionally to record an impedance distribution along the way.

By means of the scanning head of the invention it is possible to rapidly and simply scan various regions of the patient's body and to examine them as to anomalous impedance values or changes. In this way it is possible to even detect edemas that are present only locally, and optionally to quantify them in terms of their extension.

In order to achieve good electrode contact between scanning head and surface of the patient, a conduction agent such as a conductive gel may be employed as is known, e.g., from ultrasound checks.

The comparison with reference data for various, derived measurement values of the bioimpedance such as, e.g., muscle mass, fat mass and water inclusion, is conceivable. This concerns not only the impedance raw data.

A measurement may be performed as a spectroscopy with a multitude of frequencies or with a single frequency at 50 kHz, for instance. Moreover the results may be combined with other imaging methods such as, e.g., MRT (nuclear spin) or DEXA (Dual Energy X-Ray Absorptiometry). Corresponding controlling, storage and/or displaying or output devices may be provided. A simultaneous derivation of ECG signals or parts thereof via the electrodes already applied for the purpose of impedance measurement or employed independently of location is also possible. In such cases, local bioimpedance signals may be correlated directly with ECG signals (e.g., detection of extrasystoles). Corresponding controlling, storage and/or displaying or output devices may also be provided herefor.

Furthermore it is possible to combine a standard skinfold measurement for evaluation of the fat content with an impedance measurement by using the scanning head and/or system of the invention. In this case, a voltage measurement would take place between the skinfold calipers. Again, corresponding controlling, storage and/or displaying or output devices may be provided herefor.

By means of the scanning head, the arrangement, and the method of the invention, a local impedance measurement may advantageously also be possible between the two electrodes. The latter may amount to, or enable, a highly selective measurement across a delimited skin region.

In order to conduct the method according to the invention, the required devices and means may be provided. These may be configured for the respective purpose of the method according to the invention.

Preferably, the arrangement comprises a device which is configured for displaying results of imaging examinations taking into account the results of an ultrasound scan of the patient. This device may be a control unit and/or a unit for displaying the results, e.g., a monitor, a printer, a database, etc.

Preferably, the arrangement comprises a device which is configured for displaying results of imaging examinations by compiling results of impedance tomographies. This device may also be a control unit and/or a unit for displaying the results, e.g., a monitor, a printer, a database, etc.

### BRIEF DESCRIPTION OF THE DRAWINGS

The present invention shall in the following be explained exemplarily in more detail by making reference to the added, schematically simplified drawings, wherein:

FIG. 1a shows the conventional performance of an impedance measurement on a patient's body, and FIG. 1b shows the method of the invention by using a scanning head in accordance with the present invention.

FIG. 2 shows a lateral view of an optional configuration of the scanning head in accordance with the present invention.

FIG. 3 shows a schematically simplified measurement arrangement for the impedance measurement or for a method for deriving voltage differences on the surface of a patient's body in accordance with the present invention.

FIG. 4 shows exemplary voltage derivations made possible by the arrangement of FIG. 3.

FIG. 5 shows how a differential distance across the patient's abdomen may be obtained by rolling with a roller wheel (not shown).

FIG. 6 shows a schematically simplified arrangement according to the invention.

### DETAILED DESCRIPTION OF THE DRAWINGS

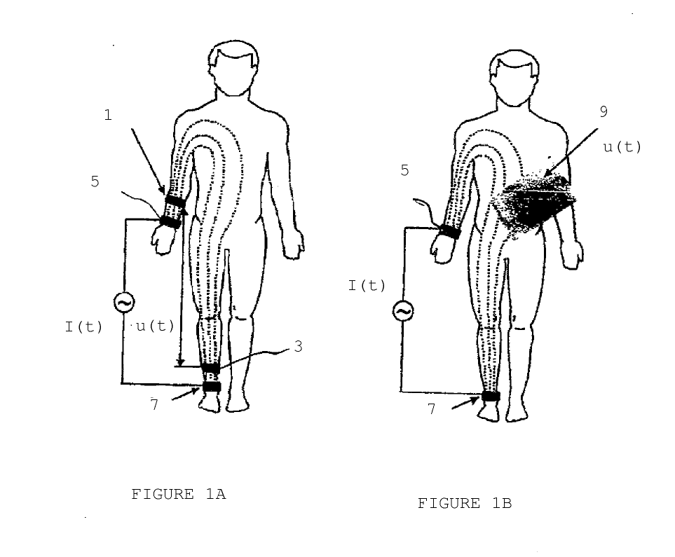

FIG. 1a shows an electrode arrangement and a current flow in a conventionally performed whole-body bioimpedance measurement which may be carried out, e.g., by means of the Fresenius Body Composition Monitor. In this case, voltage measurement electrodes 1, 3 are applied to the patient both in the region of the wrist and in the region of the ankle joint. By means of the voltage measurement electrodes 1, 3, the voltage that is induced by a current introduced into the body through the intermediary of current introduction electrodes 5, 7 is being measured. Locally increased or reduced impedances, as may occur in the leg edema or ascites exemplarily mentioned above, are not recognized in a reliable manner with this measurement arrangement. Moreover an event or condition altering the impedance can not be allocated to the afflicted portion of the patient's body.

FIG. 1b shows the impedance measurement in accordance with the present invention. To this end, a voltage induced via the current introduction electrodes 5, 7 is measured locally by means of a scanning head 9 including at least two voltage measurement electrodes (not represented). The impedance may then—just like in the prior art—be calculated in a simple manner as in the prior art. The voltage electrodes, which are not represented in FIG. 1b, are implemented inside the scanning head 9. They may, for example, have a fixed spacing from one to ten or more centimeters.

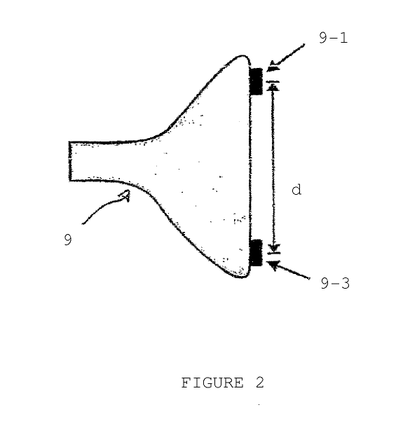

FIG. 2 shows a lateral view of an optional configuration of the scanning head 9 in accordance with the present invention including voltage measurement electrodes 9-1, 9-3. In the example of FIG. 2, these have a distance d of five centimeters from each other.

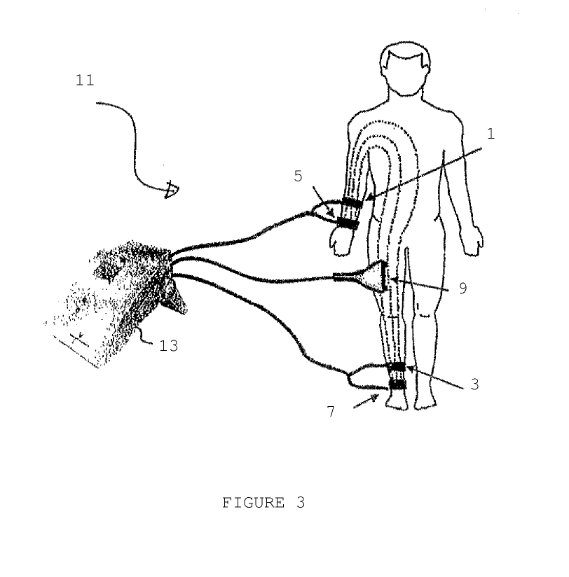

FIG. 3 is a schematically simplified representation of a measurement arrangement 11 for the impedance measurement or for the method for deriving voltage differences on the surface of a patient's body in accordance with the present invention.

The arrangement 11 of FIG. 3 includes a scanning head 9, also referred to as an impedance sensor, current introduction electrodes 5, 7 as are also represented in FIG. 1, and an impedance measurement apparatus 13. The impedance measurement apparatus 13 is connected both to the scanning head 9 and to the current introduction electrodes 5 and 7.

In the special embodiment intended to allow a voltage or impedance determination as shown in FIG. 3, the arrangement 11 furthermore includes additional voltage electrodes 1 and 3 in addition to the measurement electrodes of the scanning body 9. These electrodes 1 and 3 are purely optional and not required for carrying out the present invention. They merely constitute a possible supplementation which is equally encompassed by the present invention.

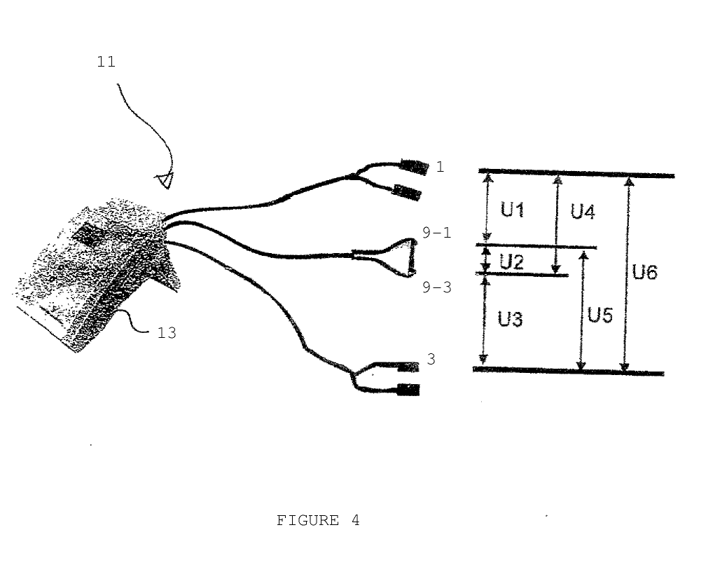

FIG. 4 shows exemplary voltage derivations enabled by the arrangement 11 of FIG. 3. Owing to the provision of altogether four voltage measurement electrodes 1, 3, 9-1 and 9-3, six different measurements are possible. This has the advantage that three independent body segments may be constructed (U1, U2, and U3). In addition, the utilization of redundancy via U4, U5, and U6 results in a possibility of advantageously reducing measurement errors. The latter results from the insight that U1+U2+U3 must equal U6.

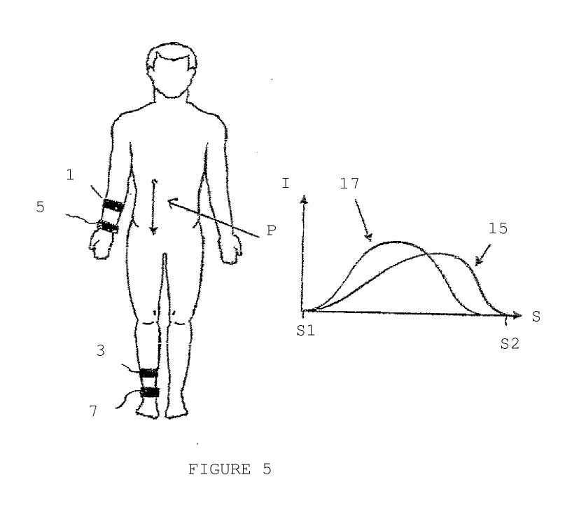

FIG. 5 shows how an impedance curve 15 may be recorded across the patient's abdomen by rolling a roller wheel (not represented) as an example of a means for determining a differential distance across the patient's abdomen. This may be compared to an impedance curve 17 of a standard population. At a corresponding deviation, e.g., the surface area indicated by the curve or the extreme values, e.g., the maximum of the impedance curve 15, it is possible to infer the presence of critical masses of visceral fat or inclusions of water.

Curves 15 and 17 of FIG. 5 represent the height of the impedance I across the distance s. In this case, s1 represents the start and s2 the end of the rolling motion P with the roller wheel across the abdomen.

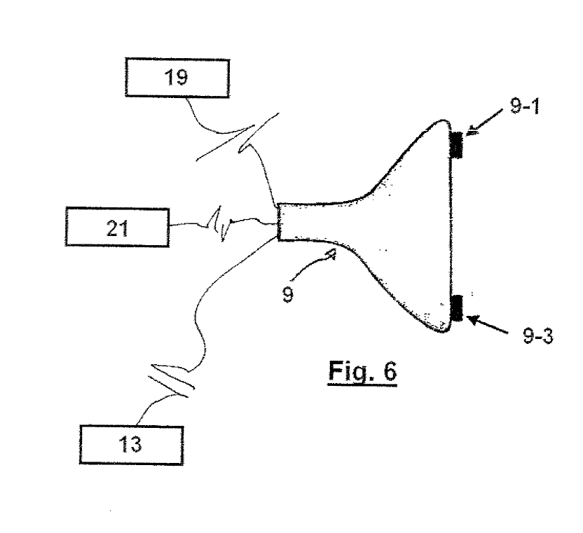

FIG. 6 shows a schematically simplified arrangement according to the invention. It optionally comprises one or more device(s), shown in FIG. 6 as bioimpedance measurement apparatus 13, a device 19 for displaying results of imaging examinations taking into account the results of an ultrasound scan of the patient, and as a device 21 for displaying results of imaging examinations by compiling or generating results of impedance tomographies. As indicated in FIG. 6, the scanning head 9 is each directly or indirectly connected with one or all of the devices 13, 19 and 21.

1-17. (canceled)

18. A scanning head comprising:
at least two electrodes, at least one electrode which is configured as a measurement electrode for the impedance measurement on a patient's body or on a portion of a patient's body.

19. The scanning head according to claim 18, wherein at least two of said electrodes are disposed on the scanning head such that during utilization of the scanning head, the two electrodes are adapted to simultaneously contact the same portion of the patient's body.

20. The scanning head according to claim 19, wherein a distance between at least two of said electrodes is fixed.

21. The scanning head according to claim 18, further comprising a means for varying the distance of at least two of said electrodes relative to each other.

22. The scanning head according to claim 21, wherein the means for varying the distance is configured as a means for shifting at least one of said electrodes relative to the scanning head, relative to another electrode, or both.

23. The scanning head according to claim 18, further comprising at least one of the following: a means for determining a position of the scanning head, a means for determining a velocity at which the scanning head is moved across the patient's surface, and a means for determining a differential distance covered by the scanning head on or above the surface of the patient's body.

24. The scanning head according to claim 23, wherein the means for determining a differential distance is configured as a roller wheel, as an optical correlation system, by means of radio sensors, or as a combination thereof.

25. An arrangement comprising:
at least one scanning head comprising at least two measurement electrodes for the impedance measurement on a patient's body or on a portion of a patient's body;
a bioimpedance measurement apparatus; and
at least two additional electrodes.

26. The arrangement according to claim 36, wherein the at least two current introduction electrodes are connected to at least two additional measurement electrodes.

27. The arrangement according to claim 25, further comprising a means for comparing measurement values to existing values.

28. The arrangement according to claim 25, further comprising a device configured for displaying results of imaging examinations taking into account the results of an ultrasound scan or ultrasound examination of the patient.

29. The arrangement according to claim 25, further comprising a device configured for displaying results of imaging examinations by compiling or generating results of impedance tomographies.

30. A method for measuring or determining impedance or changes thereof of a patient's body or part of a patient's body, comprising:
utilizing of at least two electrodes in a locally variable manner, at least one electrode which is configured as a measurement electrode.

31. A method for deriving voltages, voltage differences, or at least one electrical field or respective changes thereof on the surface of a patient's body, comprising:
utilizing of at least two electrodes in a locally variable manner, at least one electrode which is configured as a measurement electrode.

32. The method according to claim 30, further comprising:
using at least one scanning head according to claim 18 or using an arrangement according to claim 25.

33. The method according to claim 30, further comprising:
reproducing results of imaging examinations while reflecting results of an ultrasound examination of the patient.

34. The method according to claim 30, further comprising:
reproducing results of imaging examinations of the patient while generating impedance-tomographic results.

35. The scanning head according to claim 18, wherein said at least one measurement electrode comprises a voltage measurement electrode.

36. The arrangement according to claim 25, wherein the at least two additional electrodes comprise current introduction electrodes.

37. The arrangement according to claim 26, wherein said additional measurement electrodes comprise voltage measurement electrodes.

38. The method according to claim 30, wherein said at least one measurement electrode comprises a voltage measurement electrode.

39. The method according to claim 31, wherein said at least one measurement electrode comprises a voltage measurement electrode.
